(19) United States
(12) Patent Application Publication
(10) Pub. No.: US 2006/0271055 A1
(43) Pub. Date: Nov. 30, 2006

(54) Spinal stabilization

(76) Inventor: Jeffery Thramann, Longmont, CO (US)

Correspondence Address: HOLLAND & HART, LLP, P.O BOX 8749, DENVER, CO, 80201, US

(21) Appl. No.: 11/128,960

(22) Filed: May 12, 2005

(51) Int. Cl. A61B 17/56

(52) U.S. Cl. 606/74

(57) ABSTRACT

A spinal stabilization device comprises a superior anchor and an inferior anchor. An interspinous spacer extends between the anchors and provides stabilization and support. The anchors can be bands, clamps, or surfaces designed to couple the device to vertebral body parts.

### FIELD OF THE INVENTION

The present invention relates to surgical technologies and, more particularly, to methods and apparatuses for spinal stabilization.

### BACKGROUND OF THE INVENTION

For a number of years, surgical spinal correction has been tending away from conventional fusion surgical technologies to non-fusion technologies. One non-fusion technology involves using interspinous spacers. In use, a spacer is inserted into one or more spinal segments between adjacent spinous processes. An artificial ligament or, in some cases, the supraspinous ligament is used to hold the spacer in place. The artificial ligament could be, for example, nylon or polyester. The ligament inhibits migration of the spacer. The spacer is typically made out of a titanium alloy, polymers, or PEEK material. In some instances, the spacer is formed or implanted in such a way that the device has some elasticity so it can compress and expand a limited amount to accommodate movement.

Placing and securing the spacer distracts the segment in the flexed position. Thus, the spacer, among other things, opens the spinal canal, expands the neural foramen, decompresses the posterior annulus of the disc, and un-weights the facet. Thus, the spacer remove or reduces pain.

While the interspinous spacer provides several advantages, the placement surgical implant of the spacer and/or ligament is complex and difficult. Thus, it would be desirous to provide an improved method and apparatus for spinal stabilization.

### SUMMARY OF THE INVENTION

To attain the advantages and in accordance with the purpose of the invention, as embodied and broadly described herein, a spinal stabilization device is provided. The spinal stabilization device comprises a superior band and an inferior band. A spacer extends between and is coupled to the superior band and inferior band. The bands are connectable to spinous process such that the spacer and bands stabilize and support the spine.

Another embodiment of the spinal stabilization device includes a first anchor and a second anchor. The spacer extends between the anchors. At least the first anchor comprises a vertebral body engaging surface to couple the first anchor to a first lamina of a first vertebral body. The second anchor couples to the second vertebral body wherein the first vertebral body and the second vertebral body are stabilized.

Yet another embodiment of the spinal stabilization device includes a first anchor and a second anchor with a spacer extending therebetween. The first anchor comprising a first leg to extend over an anterior portion of a first vertebral body and a second leg to extend over a posterior portion of the first vertebral body, the first leg and second leg having a first position to facilitate placement of the first anchor and a second position to couple the first anchor to the first vertebral body. The second anchor couples to the second vertebral body such that the vertebral bodies are stabilized.

Still another embodiment of the present invention includes a posterior part and an anterior part. The a posterior part includes a superior end, an inferior end, and a bridge. The ends are shaped to fit about superior and inferior vertebral segments, respectively. The anterior part is rotatably connected to the posterior such that the anterior part can be rotated from a first installation orientation to a second stabilization orientation. When in the second orientation, the anterior part forms a superior clamp and an inferior clamp with the posterior part about a superior vertebral body and a inferior vertebral body when rotated in the second position. The anterior and posterior parts are connected using a connector.

The foregoing and other features, utilities and advantages of the invention will be apparent from the following more particular description of a preferred embodiment of the invention as illustrated in the accompanying drawings.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 shows a superior view of a vertebral body;

FIG. 2 shows an elevation view of the vertebral body of FIG. 1;

FIG. 3 shows a superior vertebral body and an inferior vertebral body with an interspinous spacer device consistent with and embodiment of the present invention;

FIG. 4 shows the anchor 320/324 of the device 300 in more detail;

FIG. 4A shows an another embodiment of an anchor consistent with an embodiment of the present invention;

FIG. 5 shows one possible interlock between anchor 320 and spacer 324;

FIG. 5A shows another embodiment of an interspinous spacer consistent with an embodiment of the present invention;

FIG. 6 shows an alternative embodiment of an vertebral body stabilizer consistent with the present invention;

FIG. 7 shows the transition between the Lumbar and Sacrum of the spine;

FIG. 8 shows another spinal stabilization device consistent with an embodiment of the present invention

FIG. 9 shows another device consistent with an embodiment of the present invention; and

FIG. 10A-10D shows another spinal stabilization device consistent with an embodiment of the present invention.

### DETAILED DESCRIPTION

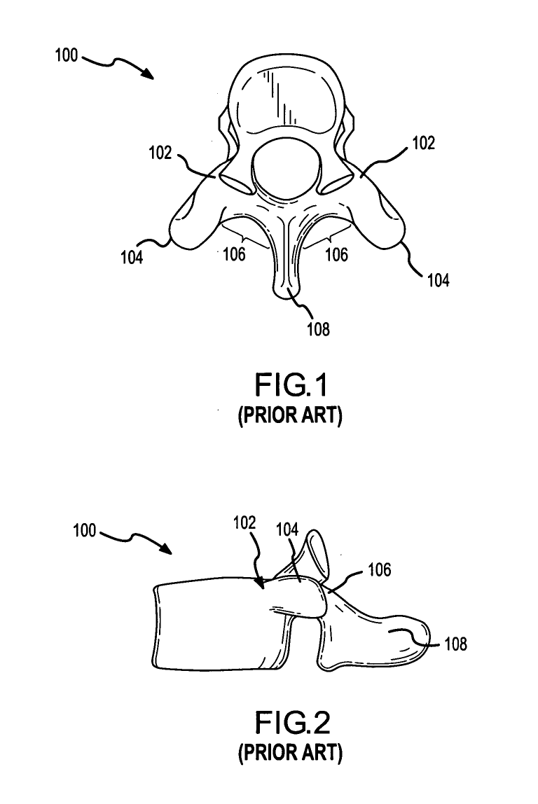

The present invention will now be described with reference to FIGS. 1 to 10. Referring first to FIGS. 1 and 2, a vertebral body 100 is shown for reference. FIG. 1 shows a superior view of a vertebral body 100 (i.e., looking down the spinal column). The vertebral body 100 comprises, among other parts, the pedicles 102, the facets 104, the lamina 106, and the spinous process 108. FIG. 2 shows a side elevation view of vertebral body 100 with a pedicle 102, the facet 104, lamina 106, and spinous process 108.

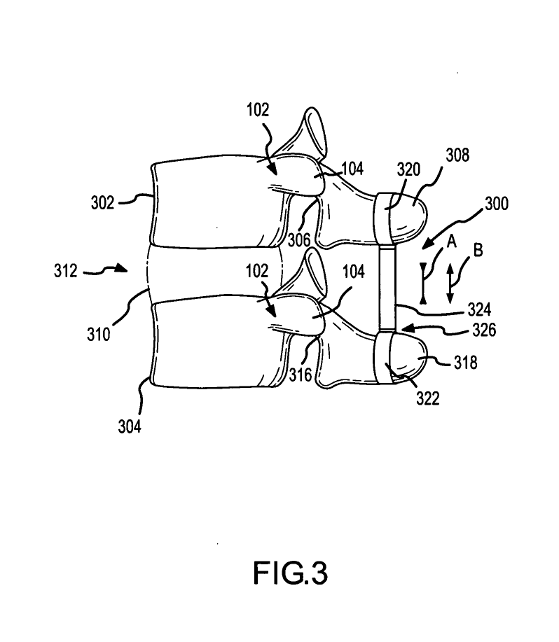

FIG. 3 shows a side elevation view of a superior vertebral body 302 and an inferior vertebral body 304 (not shown to scale and slightly exploded for ease of reference) with a spinous process spacer 300 constructed in accordance with the present invention. For reference, vertebral bodies 302 and 304 comprise the pedicle 102 and facets 104. Superior vertebral body 302 has superior lamina 306 and superior spinous process 308, and inferior vertebral body 304 has inferior lamina 316 and inferior spinous process 318. An intervertebral disk 310 typically exists in intervertebral space 312, but may be removed and/or replace by artificial discs, grafts, or the like.

Spinous process spacer 300 includes a superior anchor 320 and an inferior anchor 322 coupled to a spacer 324. Although one or the other anchor could be removed with spacer 324 abutting the spinous process or other vertebral body part on one end and being anchored on the other end. For example, superior anchor 320 may be attached to superior spinous process 308, spacer 324 attached to anchor 320 and an inferior end of spacer 324 may abut inferior spinous process 318, but not actually be anchored. Optionally, inferior end of spacer 324 may include a clamp, such as clamp 602 or 604 described below, or an engaging surface, such as surface 808 or 810 described below.

Spacer 324 is constructed out of biocompatible material, such as, for example, titanium, stainless steel, PEEK material, polymers, shaped memory alloys, or the like. Spacer 324 provides support to inhibit superior spinous process 308 from collapsing towards inferior spinous process 318, which would tend to increase pressure, collapse the neural foramen, compress the posterior annulus, and weight the facets, all of which could lead to pain generation. Spacer 324 ideally is elastic in both extension or compression (direction A) and flexion or tension (direction B) to allow for some extension and flex of the spinal column. The flexion and extension is limited to provided the necessary support. The flexion and extension could be varied by the choice of material used and the amount of support necessary. For example, in more severe degeneration cases, the movement of the spacer in direction A would be more limited to provide more support.

While spacer 324 could be constructed from a number of materials, as identified above, constructing spacer 324 out of shaped memory alloy (“SMA”) is preferred. SMAs include, for example, Nitinol (NiTi) although other SMAs could be used, such as, for example, Ag—Cd alloys, Cu—Al—Ni alloys, Cu—Sn alloys, Cu—Zn alloys, Cu—Zn—Si alloys, Cu—Zn—Sn alloys, Cu—Zn—Al alloys, In—Ti alloys, Ni—Al alloys, Fe—Pt alloys, Mn—Cu alloys, Fe—Mn—Si alloys, and the like.

Spacer 324 made from SMAs would have elasticity in both direction A and direction B. Another advantage of SMAs is that the size of spacer 324 can be altered by activation, such as, for example, by heating the SMA. Changing the size of spacer 324 could provide more or less support between superior spinous process 308 and inferior spinous process 318 depending on the amount of degeneration, other disease, and/or as healing occurs.

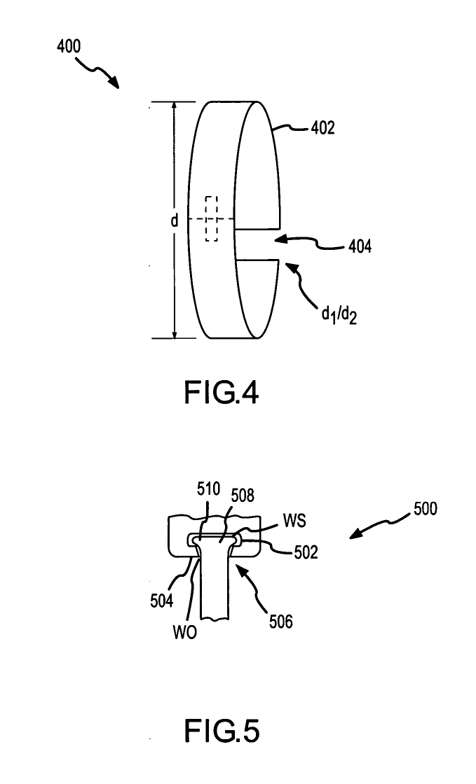

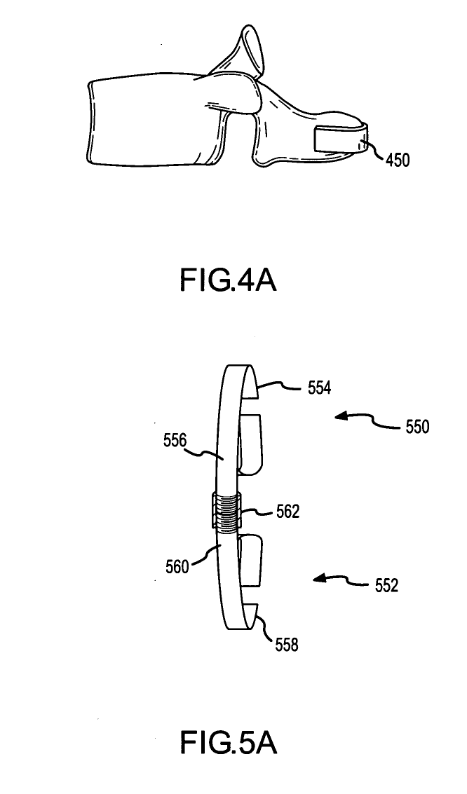

Superior anchor 320 and inferior anchor 322 couple to spacer 324 and superior spinous process 308 and inferior spinous process 318 respectively. Referring now to FIG. 4, an anchor 400 is shown in more detail. Anchor 400 comprises a band 402 or clip having gap 404. Gap 404 is provided for ease of attaching the band to spinous process 308 or 318 but could be removed such that anchor 400 is a circle, elliptical, or other shape whether geometrical or random. The shape of anchor 400 is shown generally as cylindrical, but the actual shape of anchor 400 may be designed to more conform to the actual shape of the spinous process to which it will be attached. Anchor 400 may be constructed out of any biocompatible material, such as, for example, titanium, PEEK, polymers, SMAs, or the like.

As shown, anchor 400 may comprise an elastically deformable material, such as, for example, spring metals, polymers, SMAs, or the like. To implant anchor 400, band 402 would be expanded such that gap 404 was a first size d₁ that allowed band 402 to fit about spinous process 308 or 318. Once positioned, band 402 would be allowed to contract such that gap 404 was a second size d₂ smaller than d₁ and band 402 would fit snuggly with spinous process 308 and 318. If anchor 400 was formed of SMAs, the contraction could be accomplished by activation of the metal causing it to contract to a predetermined size. Gap 404 is used relatively generically and gap 404 could be traversed by an elastic material, such as an accordion type shape, polymer, SMA, or the like.

Alternatively, anchor 400 could operate similar to a clamp. For example, a tightening device 410 (shown in phantom and comprises in this example a screw and threaded bore but could be any conventional connector as is known in the art) could be used to cause a diameter d of anchor 400 to decrease as tightening device 410 is tightened. Thus, anchor 400 would have a first, untightened position to allow for implantation and a second tightened position once implanted. Alternatively, anchor 400 could be two separate pieces connectable by tightening device 410. Whether 1 or more pieces, anchor 400 would operate in a similar manner.

Referring to FIG. 4A, an alternative anchor 450 is shown. Anchor 450 is similar to anchor 400, but instead of wrapping around the spinous process (as shown in FIG. 3) it wraps over the spinous process. Wrapping around and wrapping over are used as generic terms to distinguish the different orientations of the anchor, but the terms should not be construed to limit the invention. Anchor 450 could be fitted and attached to spinous process similar to conventional spinous process clamps associated with, for example, surgical navigation equipment.

Superior anchor 320, inferior anchor 322, and spacer 324 could be a single unit such that superior anchor 320 and inferior anchor 322 could be fitted about superior spinous process 308 and inferior spinous process 318 with spacer 324 already aligned. Alternatively, superior anchor 320, inferior anchor 322, and spacer 324 could be separate units. In this case, superior anchor 320 and inferior anchor 322 would be fitted to the respective spinous process. Spacer 324 would then be coupled to the anchors. Spacer 324 could be attached using an adhesive 326, such as, for example, a glue or thermal fusion. Alternatively, spacer 324 could be connected by an interlock 500 as shown in FIG. 5. Interlock 500 could be formed by a recess 502 with a lip 504 defining a narrow opening 506 to the recess 502 and a protrusion 508 having a shoulder 510. Shoulder 510 having a shoulder width WS larger than narrow opening width WO. The recess and protrusion could fit in a snap lock type of connection or a slidable connection such as a ridge and groove, or the like. If formed of SMAs, interlock 500 could be designed to operate similar to a clamp on activation. In other words, lip 504 would contract and clamp about shoulder 510 after activation of the memory alloy.

Alternatively, as shown in FIG. 5A, interspinous spacer 300 could comprise a superior part 550 and an inferior part 552. Superior part 550 comprises a superior anchor 554 and a superior spacer 556. Inferior part 552 comprises an inferior anchor 558 and an inferior spacer 560. Superior spacer 556 and inferior spacer 560 are coupled by a connector 562. For example, superior spacer 556 and inferior spacer 560 could be threaded and connector 562 could be a threaded sleeve or threaded bore to which the spacers thread. Other connectors as are generally known in the art are possible.

While spinous process spacer 300 works well for most vertebral bodies, one of ordinary skill in the art on reading the disclosure will now recognize, in particular, two cases where spacer 300 with two anchors (as well as conventional devices) will not work satisfactorily. The first case is where the superior spinous process 308 and/or the inferior spinous process 318 is damaged such that it cannot support the spacer 324 or anchors 320 or 322. The second case is where the spinous process simply does not exists, such as the Sacrum or S level of the spine.

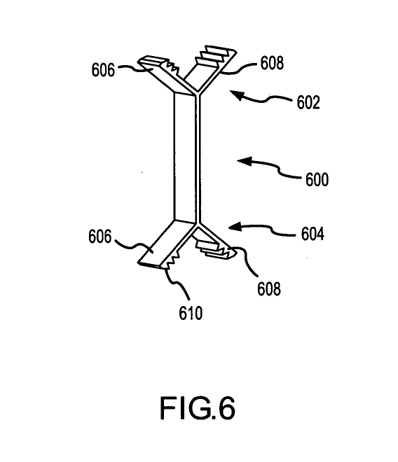

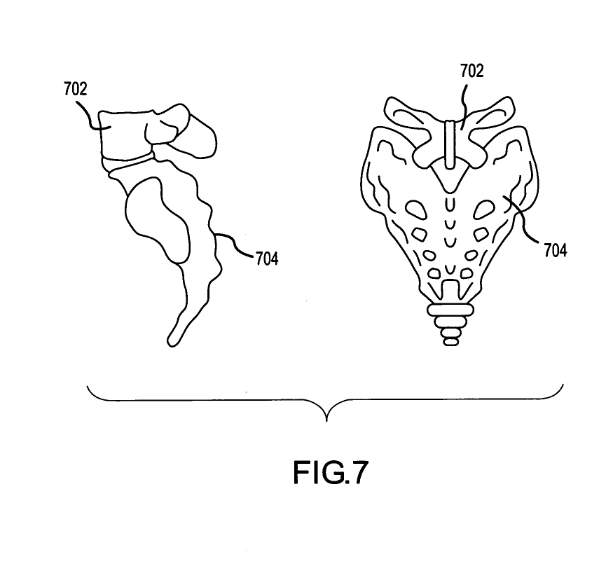

Referring to FIG. 6, an alternative spinous process spacer 600 is shown. Spacer 600 would provide at least one superior clamp 602 and at least 1 inferior clamp 604. If the clamps connected to, for example, the lamina, clamps 602 and 604 would comprise a first leg 606 on the anterior surface of the lamina and a second leg 608 on posterior side of the superior lamina. The clamps may have a protrusion 610 on the bone engaging surface of clamps 602 and 604, such as, a pin, a plurality of teeth, striations, or the like to assist clamps 602 and 604 with gripping the laminas. Instead of a superior clamp and an inferior clamp, spacer 600 may comprise two or more superior clamps and two or more inferior clamps such that the spacer is symmetrically supported by the vertebral bodies. Also, instead of clamping to the laminas, clamps 602 and 604 may clamp about superior and inferior spinous process. Moreover, anchors, such as, superior and inferior anchors 320 and 322 could be used with superior and inferior clamps 602 and 604 depending on the patient anatomy. Moreover, the combination could be used for the transition between the 5th lumbar vertebral body 702 and sacrum 704 shown in FIG. 7. Spacer 600 could similarly be 2 or more parts as explained above.

Spacer 300, spacer 600, or some combination thereof, could also be used to support multiple levels of vertebral bodies after, for example, a vertebral body removal or a portion of a vertebral body removal. For example, a spinous process and lamina of a middle vertebral body is surgically removed, spacer 300, spacer 600, or some combination could be used to provide artificial skeletal like support between the outer superior and inferior vertebrae. In other words, the spacers could be used as a bridge over multiple levels of vertebral segments by providing support and stabilization.

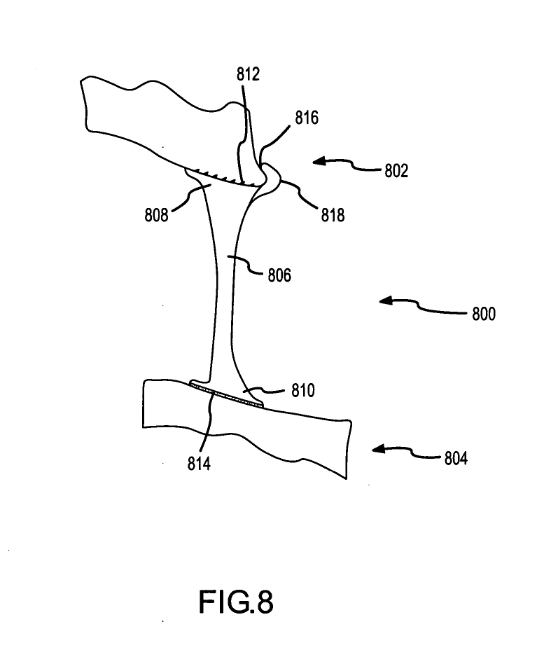

Referring now to FIG. 8, a lamina column space 800 is shown. Lamina column spacer 800 functions similar to spinous process stabilization spacers (such as 300 and 600 above), but does not connect to the spinous process. Lamina column spacer 800 extends from a superior lamina 802 to an inferior lamina 804. Lamina column spacer 800 includes a column support 806 and a superior lamina engaging surface 808 and an inferior lamina engaging surface 810. Engaging surfaces 808 and 810 may be enlarged, flanged, or flared as shown to provide a larger surface area to connect with the lamina portion of the vertebral body. Engaging surfaces 808 and 810 may have ridges, protrusions, striations, or the like (as represented by reference number 812) to increase the frictional lock between the surfaces and the lamina. Alternatively or in combination with, an adhesive 814 may reside between surfaces 808 and 810 and the lamina. Adhesive 814 may be a glue, a bone growth factor, or the like. Lamina column spacer 800 may be constructed out of any biocompatible material, such as, for example, titanium, stainless steel, polymers, SMAs, or the like. Engaging surfaces 808 and 810 may reside substantially adjacent an edge 816 of lamina, such as engaging surface 808 is about an edge 816 of superior lamina 802. If arranged on the edge 816, the engaging surface may have wrap, lip or groove (as represented by reference number 818) that curls around edge 816. Notice, while engaging surfaces 808 and 810 are described as engaging the lamina portion of the vertebral bodies, one of ordinary skill in the art will know recognize that the lamina engaging surfaces could engage and relatively flat portion of the vertebral body to form the wedge or friction lock for the spacer. Engaging surfaces 808 and 810 could be used in combination with anchors 320 and 322 or clamps 602 and 604.

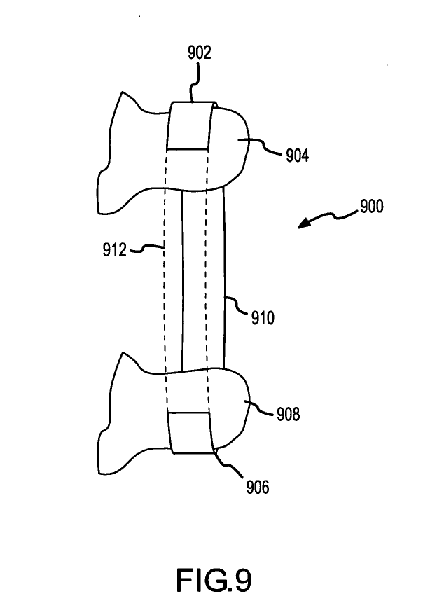

To provide greater resistance to flex, an enlarged band 900 may be used to inhibit motion, see FIG. 9. Enlarged band 900 has a superior loop 902 that loops or hooks around superior spinous process 904 and an inferior loop 906 that loops or hooks around inferior spinous process 908. As shown, enlarged band 900 is one sided 910 and forms a generally C shape. However, enlarged band could be a complete circle or elliptical shape by including a second side 912 shown in phantom. Moreover, spacer 324, 600, or 800 could be integrated with enlarged band 900.

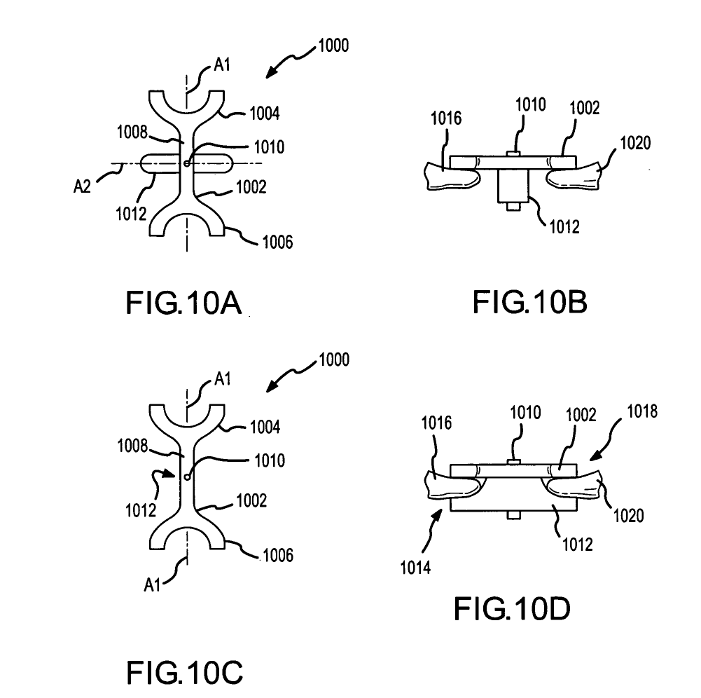

Referring to FIGS. 10A, 10B, 10C, and 10D, an interspinous process device 1000 is shown. Device 1000 has a posterior part 1002 comprising a superior end 1004 having a generally V, Y U or C shape and an inferior end 1006 having a generally V, Y, U or C shape, which shapes are exemplary and non-limiting. The shape of end 1004 and end 1006 are largely defined by the anatomy of the patient. A bridge 1008 extends between superior end 1004 and inferior end 1006, forming a generally H like shape. Bridge 1008 has a longitudinal axis A1. Bridge 1008 has a connector 1010 extending from posterior part 1002 to an anterior part 1012. Anterior part 1012 has a longitudinal axis A2. Parts 1002 and 1012 are referred to as posterior and anterior for convenience and should not be considered limiting. Connector 1010 is rotatably coupled to bridge 1008 and fixedly connected to anterior part 1012 such that connector 1010 and anterior part 1012 can rotate with respect to bridge 1008. As shown in FIG. 10A, anterior part 1012 has a first position generally such that axis A1 is generally perpendicular to axis A2. As shown in FIG. 10C, rotating connector 1010 rotates anterior part 1012 such that axis Al is generally parallel to axis A2. When rotated into the second position, superior end 1004 and anterior part 1012 form a clamp 1014 about superior vertebral body 1016, and inferior end 1004 and anterior part 1012 form a clamp 1018 about inferior vertebral body 1020.

While the invention has been particularly shown and described with reference to one or more embodiments thereof, it will be understood by those skilled in the art that various other changes in the form and details may be made without departing from the spirit and scope of the invention.

I claim:

1. A spinal stabilization device, comprising:
a superior band to couple to a superior spinous process;
an inferior band to couple to an inferior spinous process; and
a spacer, the spacer coupled to the superior band and the inferior band, wherein a superior vertebral body and an inferior vertebral body are stabilized and the spacer inhibits flexion and extension.

2. The spinal stabilization device of claim 1, wherein at least one band selected from the group of bands consisting of the superior band and the inferior band comprise at least one gap such that the band can be expanded and contracted to facilitate implanting the band on the interspinous process.

3. The spinal stabilization device of claim 1, wherein the spacer comprises at least a superior part and an inferior part connected by a connector.

4. The spinal stabilization device of claim 3, wherein the connector is a threaded sleeve.

5. The spinal stabilization device of claim 1, wherein the superior band, inferior band, and the spacer comprise a single integrated device.

6. The spinal stabilization device of claim 1, further comprising a plurality of interlocks, the plurality of interlocks coupling the spacer to the superior band and the inferior band.

7. The spinal stabilization device of claim 6, wherein each of the plurality of interlocks comprise a recess and a protrusion.

8. The spinal stabilization device of claim 1, wherein the superior band and the inferior band comprise at least one shaped memory alloy and the at least one shaped memory alloy has a first shape to facilitate implant and a second shape to fit snuggly about the spinous process.

9. The spinal stabilization device of claim 1, wherein the spacer comprises at least one shaped memory alloy.

10. A spinal stabilization device, comprising:
a first anchor;
a second anchor; and
a spacer extending between the first anchor and the second anchor;
at least the first anchor comprising a vertebral body engaging surface to couple the first anchor to a first lamina of a first vertebral body; and
the second anchor to couple to a second vertebral body, wherein the first vertebral body and the second vertebral body are stabilized.

11. The spinal stabilization device of claim 10, wherein the second anchor comprises a vertebral body engaging surface to couple the second anchor to a second lamina of the second vertebral body.

12. The spinal stabilization device of claim 11, wherein the vertebral body engaging surface is at least one of enlarged, flanged, or flared.

13. The spinal stabilization device of claim 10, wherein the vertebral body engaging surface is formed to wrap around an edge of the first vertebral body.

14. The spinal stabilization device of claim 10, wherein the second anchor comprises a band to fit about the spinous process of the second vertebral body.

15. The spinal stabilization device of claim 10, wherein the vertebral body engaging surface comprises at least one surface treatment to facilitate a frictional engagement between the vertebral body engaging surface and the first vertebral body.

16. The spinal stabilization device of claim 15, wherein the at least one surface treatment comprises a surface treatment selected from a group of surface treatments consisting of: ridges, protrusions, striations, or adhesives.

17. A spinal stabilization device, comprising:
a first anchor;
a second anchor; and
a spacer extending between the first anchor and the second anchor;
at least the first anchor comprising a first leg to extend over an anterior portion of a first vertebral body and a second leg to extend over a posterior portion of the first vertebral body, the first leg and second leg having a first position to facilitate placement of the first anchor and a second position to couple the first anchor to the first vertebral body; and
the second anchor to couple to the second vertebral body, wherein first and second vertebral bodies are stabilized.

18. The spinal stabilization device of claim 17, wherein the second anchor comprises a vertebral body engaging surface.

19. The spinal stabilization device of claim 17, wherein the second anchor comprises a band

20. The spinal stabilization device of claim 17, wherein the second anchor comprises a third leg to extend over an anterior portion of a second vertebral body and a fourth leg to extend over a second vertebral body, the first leg and second leg having a first position to facilitate placement of the first anchor and a second position coupling the first anchor to the first vertebral body.

21. The spinal stabilization device of claim 17, wherein the first leg and the second leg couple to a lamina of the first vertebral body.

22. A spinal stabilization device, comprising:
a posterior part, the posterior part comprising:
a superior end;
an inferior end; and
a bridge,
wherein the superior end is shaped to fit about a superior vertebral segment and the inferior end is shaped to fit about an inferior vertebral segment, and the bridge extends between the superior end and the inferior end;
an anterior part, the anterior part being rotatable with respect to the posterior part between a first position and a second position, wherein the anterior part forms a superior clamp and an inferior clamp with the posterior part about a superior vertebral body and a inferior vertebral body when rotated in the second position; and
a connector connecting the posterior part and the anterior art.

23. The spinal stabilization device of claim 22, wherein the superior vertebral segment is a spinous process.

24. The spinal stabilization device of claim 22, wherein the inferior vertebral segment is a spinous process.

25. The spinal stabilization device of claim 22, wherein the inferior vertebral body is a S-level body.

26. The spinal stabilization device of claim 1, wherein at least one of the superior band and the inferior band further comprises a tightening device.

27. The spinal stabilization device of claim 26, wherein the tightening device comprises a screw and threaded bore.

28. The spinal stabilization device of claim 26 wherein at least one of the superior band and the inferior band comprise a plurality of parts coupled by the tightening device.

29. A spinal stabilization device, comprising:
a first band coupled to a first vertebral body; and
a spacer coupled to the first band; and
an anchor engaged with a second vertebral body, wherein the first vertebral body and the second vertebral body are stabilized and the spacer inhibits flexion and extension.

30. The spinal stabilization device of claim 29, wherein the anchor comprises a first leg and a second leg, wherein the first leg and the second leg are arranged on opposite sides of a spinous process.

31. The spinal stabilization device of claim 29, wherein the anchor comprises a first leg and a second leg, wherein the first leg and the second leg are arranged on opposite sides of a lamina.

32. The spinal stabilization device of claim 30, wherein the first leg has at least a first protrusion and the second leg has a least a second protrusion.

33. The spinal stabilization device of claim 30, wherein the first leg and the second leg are constructed out of a shaped memory alloy.
